(19) United States
(12) Patent Application Publication
(10) Pub. No.: US 2020/0160881 A1
(43) Pub. Date: May 21, 2020

(54) LANGUAGE DISORDER DIAGNOSIS/SCREENING

(71) Applicant: Therapy Box Limited, Warple Way (GB)

(72) Inventors: Swapnil Ravindra Gadgil, Wellesley (GB); Rebecca Louise Bright, Wellesley (GB)

(21) Appl. No.: 16/659,597

(22) Filed: Oct. 22, 2019

(30) Foreign Application Priority Data
Nov. 15, 2018 (GB) 18186346

(51) Int. Cl. G10L 25/66; G06N 20/00; G10L 15/26; G10L 15/00; A61B 5/00

(52) U.S. Cl. CPC G10L 25/66; G06N 20/00; A61B 5/4803; G10L 15/005; G10L 15/265

(57) ABSTRACT

Language disorder diagnostic/screening methods, tools and software are provided. Audio data including speech of a subject is received. The audio data is transcribed to provide text data. Speech and language features are extracted from the text data and from the audio data. The extracted features are evaluated using a classification system to diagnose/screen whether the subject has a language disorder. The classification system includes at least one machine learning classifier. A diagnosis/screening is output.

### FIELD OF THE INVENTION

The present invention generally relates to language disorder diagnosis/screening methods, tools and software, and more particularly relates to language disorder diagnosis or screening making use of machine learning evaluation of speech of a subject to diagnose or screen a language disorder.

### BACKGROUND ART

A language disorder is an impairment that makes it hard for a subject to find the right words and form clear sentences when speaking. It can also make it difficult for the subject to understand what another person says. A subject may have difficulty understanding what others say, may struggle to put thoughts into words, or both.

One example of a language impairment is developmental language disorder (DLD). Development Language Disorder (DLD) is defined as a condition when children have a delay in acquiring skills related to language for no obvious reason. Children diagnosed with DLD may have difficulty with educational and social attainment which can serve as a major impediment later on in their life. Sometimes difficulties learning language are part of a broader developmental condition, such as autism or Down syndrome. For others, language deficits are unexplained, and other aspects of development may not be so affected. As a community, we have agreed to identify these children as having Development Language Disorder, or DLD.

Diagnosing and treating language disorders at early stages is imperative. However, previous and current therapeutic practices are prone to human error and are time consuming.

Accordingly, it is desirable to provide tools and methods to assist in the diagnosis/screening of language disorders. In addition, it is desirable to increase time efficiency and consistency of accuracy of language disorder diagnosis/screening. Furthermore, other desirable features and characteristics of the present invention will become apparent from the subsequent detailed description of the invention and the appended claims, taken in conjunction with the accompanying drawings and the background of the invention.

### SUMMARY

A language disorder diagnostic/screening method is provided. The method includes receiving audio data including speech of a subject, transcribing, via at least one processor, the audio data to provide text data, extracting, via at least one processor, speech and language features from the text data and from the audio data, evaluating the extracted features using a classification system to diagnose/screen whether the subject has a language disorder, and outputting the diagnosis/screening. The classification system includes at least one machine learning classifier.

This approach uses efficient combinatorial machine learning solutions to diagnose/screen whether a subject has a language disorder. The claimed subject matter reduces diagnosis/screening wait times and mitigates human error through the use of machine learning algorithms.

In embodiments, the language disorder is Developmental Language Disorder.

In embodiments, the classification system includes a plurality of classifiers. The method includes combining, via at least one processor, classification outputs from each of the plurality of classifiers. In embodiments, classification outputs from each of the plurality of classifiers is combined using a different weighting.

In embodiments, the classification system includes a random forest classifier. In embodiments, the classification system includes a convolution neural network. In embodiments, the classification system includes a linear regression classifier.

In embodiments, at least one of the classifiers operates on a spectrogram of the audio data (rather than the extracted features).

In embodiments, the classification system includes at least two of a random forest classifier, a linear regression classifier and a convolutional neural network. In embodiments described herein, where one of two classifiers (or one or two of three classifiers) might fail or err, the classification system is still capable of outputting a result.

In embodiments, the method includes transforming, via at least one processor, the audio data into a spectrogram and evaluating the extracted features and the spectrogram using the classification system to diagnose/screen whether the subject has a language disorder. In embodiments, the spectrogram is generated by transforming the audio data, which is in time domain, into frequency domain such as through a Fourier transform.

In embodiments, the method includes pre-processing of the audio data prior to extracting speech features, wherein pre-processing comprises at least one of denoising and speaker separation. In embodiments, speaker separation includes separating the speech of the subject from another person's speech, such as speech of child subject from speech of an adult.

In embodiments, the extracted features includes at least one of audio features, acoustic features and mapping features derived from the text data. In embodiments, the audio features include features based on speaker utterances and pauses.

In embodiments, the mapping features include grammar characteristics and keyword related features. Keywords can be identified by comparing words of the text data with a reference list of keywords. In embodiments, the audio features include length of speech and speech fluency related features. In embodiments, the audio features include at least one of number of pauses in the audio data, number of pauses per minute in the audio data, maximum length of utterances in the audio data, average length of utterances in the audio data, total length of time of speech of the subject in the audio data, maximum length of a pause in the audio data, ratio of maximum length of a pause and total length of time of speech of subject in the audio data, average length of pauses in the audio data, ratio of average length of pauses and total speech length, number of pauses having a length greater than five seconds in the audio data, number of pauses having a length greater than ten seconds in the audio data, the number of pauses per minute greater than ten seconds.

In embodiments, the acoustic features are extracted from a spectrogram of the audio data. In embodiments, the acoustic features include at least one of loudness, pitch and intonation of the speech of the subject.

In embodiments, the mapping features include at least one of synonyms to story keywords, a count of the number of unique synonyms achieved for each word divided by the total number of words, a count of the number of unique synonyms achieved for each word, a ratio representing how many plural words were used in a sentence, number of story keywords that were detected, a ratio representing how many pronouns were used per sentence, a ratio representing how many present progressive phrases were used per sentence, a measure of how cohesive the sentence is based on subjective and dominant clauses, a ratio that indicates how many words are incorrectly spelled in the text data, a count of the number of unique words that appeared in the list, a ratio that indicates how many different unique words were used per sentence, the ratio of the words: and/or in the document, a ratio that indicates how many low frequency words were used in the sentence, a count of the number of subordinate clauses that were used in the sentence, a ratio that indicates how many subordinate clauses were used in the sentence, a total number of words in the text data, an average number of words per utterance in the text data.

In another aspect, the language disorder diagnosis/screening tool, comprises at least one processor configured to receive audio data including speech of a subject, at least one processor configured to transcribe the audio data to provide text data, at least one processor configured to extract speech features from the text data and from the audio data, and a classification system configured to evaluate the extracted features to diagnose/screen whether the subject has a language disorder, and at least one processor configured to output the diagnosis/screening. The classification system includes at least one machine learning classifier.

In embodiments, the audio data is recorded on a user device such as a mobile phone, a laptop, a tablet, a desktop computer, etc. In embodiments, the output of the diagnosis/screening is output to a user device, e.g. a display thereof. In embodiments, the classification system is at a server remote from a user device or the classification system is at the user device. In embodiments, the audio data is transmitted over a network from the user device to the remote server.

The features of the method aspects described herein are applicable to the diagnosis/screening tool and vice versa.

In another aspect, at least one software application is configured to be run by at least one processor to cause transcribing of received audio data to provide text data, extracting speech and language features from the text data and from the audio data, evaluating the extracted features using a classification system to diagnose/screen whether the subject has a language disorder, and outputting the diagnosis/screening. In embodiments, the classification system includes at least one machine learning classifier.

The features of the method aspects described herein are applicable to the diagnosis/screening tool and vice versa.

### BRIEF DESCRIPTION OF THE DRAWINGS

The present invention will hereinafter be described in conjunction with the following drawing figures, wherein like numerals denote like elements, and

FIG. 1 is a schematic diagram of a system for language disorder diagnosis/screening, in accordance with various embodiments;

FIG. 2 is a schematic diagram of a language disorder diagnostic/screening tool, in accordance with various embodiments;

FIG. 3 is a schematic diagram illustrating training of machine learning classifiers of a language disorder diagnostic/screening tool, in accordance with various embodiments; and

FIG. 4 is a flowchart illustrating a method of language disorder diagnosis/screening, in accordance with various embodiments.

### DETAILED DESCRIPTION

The following detailed description is merely exemplary in nature and is not intended to limit the invention or the application and uses of the invention. Furthermore, there is no intention to be bound by any theory presented in the preceding background or the following detailed description.

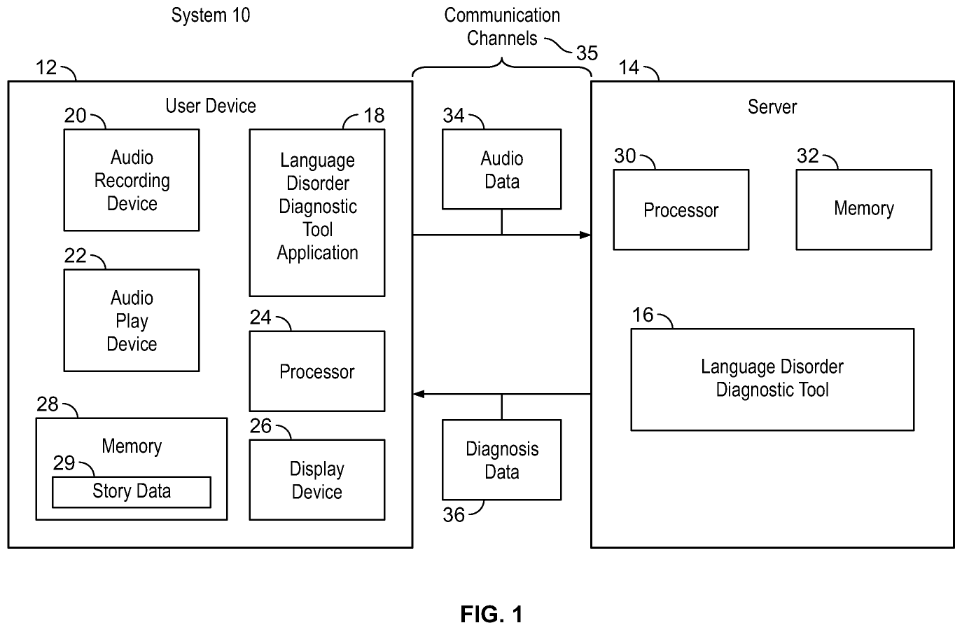

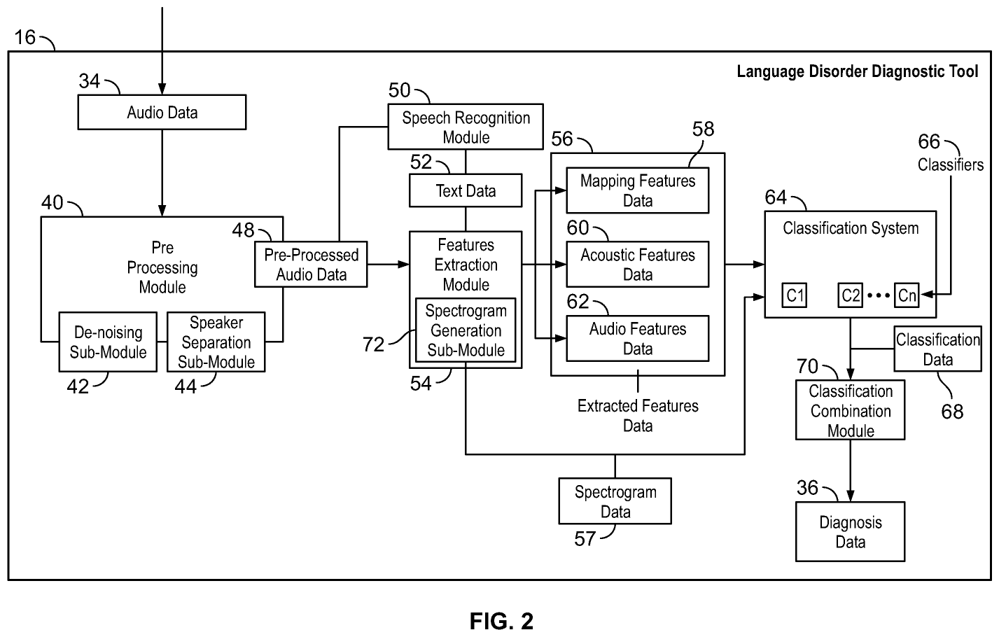

FIG. 1 is a representation of a system for language disorder diagnosis/screening, LDS, according to various embodiments. FIG. 2 is a schematic diagram of an LDS tool 16 used in the system 10, in accordance with various embodiments. In embodiments, and with reference to FIGS. 1 and 2, the system 10 generates audio data 34 recorded from speech of a subject, processes the audio data 34 using a LDS tool 16 that includes one or more machine learning classifiers and outputs a language disorder diagnosis/screening. In embodiments, the LDS tool 16 is configured to execute pre-processing stages on the audio data 34 including pre-processing of the audio data 34 to produce pre-processed audio data 48 (see FIG. 2) in which a subject's speech and sounds have been separated from sound and speech of any others and in which background noise has been filtered out. The LDS tool 16 is configured to transcribe the pre-processed audio data to provide text data 52 and to extract features from both the pre-processed audio data 48 and the text data 52 to provide extracted features data 56. A classification system 64 including at least one machine learning classifier takes the extracted features data 56 and outputs one or more classification results in the form of classification data 68. The LDS tool 16 is configured to output diagnosis/screening data 36 representing a language disorder diagnosis/screening based on the classification data 68.

Referring to FIG. 1, the system 10 includes a user device 12 and a server 14. The user device 12 includes an LDS tool application 18, which is embodied by software stored on memory 28 and executed by processor 24. The user device 12 is, in various embodiments, a mobile device, a tablet device, a laptop computer, a desktop computer, etc. The LDLT application 18 is, in some embodiments, downloaded to user device 12 from server 14 over communication channels 70. Communication channels 70 include internet and other far communication systems.

Continuing to refer to FIG. 1, the LDS tool application 18 is configured to generate audio data 34 including speech and sounds from the voice of a subject. The LDS tool application 18 is, in embodiments, configured to utilize an audio recording device 20, e.g. a microphone, of the user device 12 in order to record the speech of the subject and to generate an audio file providing the audio data 34. In embodiments, the LDS tool application 18 is configured to generate a graphical user interface for display on a display device 26 of the user device 12. The graphical user interface includes prompts for inputs from a user including subject name and other user registration data. In embodiments, the LDS tool application 18 is configured to access story audio data 29 or other pre-recorded audio information stored in memory 28 or accessed from memory 32 of server 14 or accessed from another remote server. The story audio data (or other pre-recorded audio data) 29 is played to the subject through the audio play device 22, e.g. one or more speakers. The LDS tool application 18 is configured, through graphical user interface and/or through the audio play device 22, to either output questions about the played story audio data 29 or to prompt the subject to retell, in their own words, the played story audio data 29. The answers/retelling from the subject is recorded by the audio recording device 20, thereby providing the audio data 34.

In various embodiments, the subject is a child and will usually be supervised by one or more adults including, optionally, a parent or a medical professional (such as a speech therapist). In embodiments, the language disorder is developmental language disorder.

In the example system 10 of FIG. 1, the user device 12 is configured to send the audio data 34 to the server 14 over communications channels 70 for further processing and diagnosis/screening through a remote, server-based LDS tool 16. In other embodiments, the LDS tool 16 is located at the user device 12, e.g. as part of the language disorder diagnostic/screening tool application 18. Other distributions of audio data gathering and LDS data processing capabilities than those presented herein are envisaged.

In FIG. 1, the server 14 includes processor 30, memory 32 and software for implementing the LDS tool. In embodiments, the server 14 is configured to interact with many user devices over communications channels 70. Exemplary interactions include sending the LDS tool application upon request, sending diagnosis/screening data 36 and receiving audio data 34. Diagnosis/screening data 36 includes a diagnosis/screening result representing whether a subject has a language disorder as part of an output of the LDS tool 16. In embodiments, the LDS tool application 18 is configured to present the diagnosis/screening result to a user through the audio play device and/or the display device 26. The presentation of the diagnosis/screening result may be accompanied by a recommendation for further action when a positive diagnosis/screening is received such as a recommendation to seek further advice from a language disorder professional (such as a speech therapist). The processing of the LDS tool on the processor 30 of the server 14 is discussed further herein with respect to FIG. 2.

FIG. 2 is a schematic illustration of the LDS tool 16, in accordance with various embodiments. The LDS tool 16 is described with reference to module and sub-modules thereof. As used herein, the term module refers to any hardware, software, firmware, electronic control component, processing logic, and/or processor device, individually or in any combination, including without limitation: application specific integrated circuit (ASIC), an electronic circuit, a processor (shared, dedicated, or group) and memory that executes one or more software or firmware programs, a combinational logic circuit, and/or other suitable components that provide the described functionality. Generally, the modules and sub-modules disclosed herein are executed by at least one processor 24, 30, which is included in one or more of the user device 12 and the server 14. It will be understood that modules and processing described herein can be alternatively sub-divided or combined and otherwise distributed. The shown and described arrangement of modules is merely by way of example and for ease of understanding. Any other combination of software modules can be provided for configuring the respective processors to implement the described processing functionality.

In the illustrated embodiment of FIG. 2, audio data 34 is received at the LDS tool 16, which has been generated through the audio recording device 20 of the user device 12. A pre-processing module 40 is configured to denoise the audio data via a denoising sub-module 42 and to separate subject speech and sounds (from unwanted speech and sounds) via the speaker separation sub-module 44, thereby providing pre-processed audio data 48. In some embodiments, the denoising sub-module is configured to use a fast fourier transform to remove noise, e.g. background noise such as children crying, screaming, from the audio data 34. For example, a spectral subtraction algorithm could be used. In one example, spectral subtraction is used to remove noise from noisy speech signals in the audio data 34 in the frequency domain. This exemplary method includes computing the spectrum of the noisy speech audio data 34 using the Fast Fourier Transform (FFT) and subtracting the average magnitude of the noise spectrum from the noisy speech spectrum. A noise removal algorithm can be implemented using Python software by storing the noisy speech data into Hanning time-widowed half-overlapped data buffers, computing the corresponding spectrums using the FFT, removing the noise from the noisy speech, and reconstructing the speech back into the time domain using the inverse Fast Fourier Transform (IFFT).

In embodiments, the speaker separation sub-module 44 is configured to receive the denoised audio data 34 and to separate a subject's speech from any other speakers. In embodiments where the subject is a child, one or more adult other speakers may be included in the audio data 34 (such as a parent of the child). The speaker separation sub-module 44 is configured to execute a speaker diarization algorithm that has been trained on female/male adult speakers to allow child and adult speakers to be separated so that any adult speaker audio can be removed. An exemplary algorithm includes steps of dividing the audio data into audio data segments and obtaining Mel Frequency Cepstral Coefficents (MFCCs) for each segment. Using a model of MFCCs for male and female adult speakers, probabilities are added to each segment that the respective audio segments belong to male and female adult speakers, thereby allowing the likelihood of adult speakers for audio segments to be determined and such audio segments removed. Although a model for male and female adult speakers is exemplified, it is envisaged that evolved models can be used with increasing amount of recorded children audio. Also, the recognition of children audio will start to be possible based on a model that represents children's characteristics.

The pre-processed audio data 48, in which noise and non-subject speaker's audio, has been removed is used as an input for speech recognition module 50 and features extraction module 54. Speech recognition module 50 is configured to transcribe the pre-processed audio data 48 to obtain text data 52. The text data 52 is utilized as an input to the features extraction module 54. The speech recognition module 50 is configured to employ a speech to text algorithm. The speech recognition module 50 is configured to operate on the pre-processed audio data 48, or power spectrums obtained therefrom or MFCCs obtained. A number of speech to text algorithms are available including from Google®, and IBM's Watson. In embodiments, the speech recognition module 50 is an end-to-end model for speech recognition which combines a convolutional neural network based acoustic model and a graph decoding which is based on a known speech recognition system called wav2letter. The algorithm of the speech recognition module 50 is trained using letters (graphemes) directly. In other words, it is trained to output letters, with transcribed speech, without the need for force alignment of phonemes. The model is trained on a plurality of speech libraries including children audio recordings.

Continuing to refer to FIG. 2, the features extraction module 54 is configured to extract features based on both the text data 52 and the pre-processed audio data 48. In embodiments at least three classes of features are, algorithmically, extracted via the features extraction module 54 and included in extracted features data 56. In embodiments, the at least three classes of features include audio features (audio raw characteristics), acoustic features (physical properties of audio) and mapping features (language, vocabulary and grammar). The features extraction module 54 is configured to output extracted features data including acoustic features data 60 corresponding to the acoustic features, audio features data 62 corresponding to the audio features and mapping features data 58 corresponding to the mapping features.

In embodiments, audio features are directly extracted from the pre-processed audio data 48. In some embodiments, audio features focus on utterances (the times that a person speaks) and pauses in the pre-processed audio data 48. In embodiments, audio features include number and length of pauses in speech of the subject and number and length of utterances in speech of the subject, as derived from the pre-processed audio data 48.

In various embodiments, mapping features are directly analyzed from the text data 52. Mapping features include, in embodiments, word features mapped from the text data 52. Grammar and vocabulary features derivable from words included in the text data 52 form part of the mapping features. For example, features are extracted representing a variety of language in the text data 52 (number of different words, use of synonyms), a sophistication of language in the text data 52 (based on length of words) and language comparison with reference text data corresponding to the story played to the subject which is being retold.

In embodiments, the features extraction module 54 includes a spectrogram generation sub-module 72 configured to generate a spectrogram and output corresponding spectrogram data 57. The spectrogram generation sub-module 72 is configured to generate a spectrogram from the pre-processed audio data that includes three dimensions, namely frequency, time and amplitude of a particular frequency at a particular time. In one embodiment, the spectrogram is generated using a Fourier transform. A spectrogram using a fast Fourier transform is a digital process, whereby the pre-processed audio data 48, in the time domain, is digitally sampled and the digitally sampled data is broken up into segments, which usually overlap. The segments are Fourier transformed to calculate the magnitude of the frequency spectrum for each segment. Each segment corresponds a measurement of magnitude versus frequency for a specific moment in time (the midpoint of the segment). In embodiments, the features extraction module 54 is configured to analyze the spectrogram to obtain acoustic feature values.

The features extraction module 54 is configured to receive spectrogram data 57 from the spectrogram generation sub-module 72 and to extract acoustic features for inclusion in acoustic features data 60 based on the spectrogram data 57. Exemplary acoustic features include loudness, pitch and intonation.

The features extraction module 54 is configured to output values for exemplary audio features as shown in the table below. It should be appreciated that any number and any combination of such features could be extracted. Audio features are those that have been derived from the pre-processed audio data 48.

| Audio Feature | Description |
|---|---|
| Number_Pauses | The number of pauses throughout the pre-processed audio data 48 |
| Number_Pauses_Ratio_per_min | The number of pauses per minute |
| Max_Length_Utterances | The maximum length of an utterance in the pre-processed audio data 48 |
| Mean_Length_Utterances | The average length of an utterance in the pre-processed audio data 48 |
| Subject_Speech_Duration | The total length of time that the subject spoke |
| Subject_Speech_Duration_Ratio | The length of the time that the subject spoke divided by the total length |
| | of the pre-processed audio data 48 |
| Duration | The length of the pre-processed audio data 48 |
| Max_Length_Pause | The maximum length of a pause during the pre-processed audio data 48 |
| Max_Length_Pause_Ratio | The maximum length of a pause during the pre-processed audio data 48 |
| | divided by the total speech length |
| Mean_Length_Pauses | The average length of a pause in the pre-processed audio data 48 |
| Mean_Length_Pauses_Ratio | The average length of a pause in the pre-processed audio data 48 |
| | divided by the total speech length |
| Nb of pauses sup 5 | The number of pauses whose length was greater than 5 seconds |
| Nb of pauses sup | The number of pauses whose length was greater than 5 seconds per |
| 5_Ratio_per_min | minute |
| Nb of pauses sup 10 | The number of pauses whose length was greater than 10 seconds |
| Nb of pauses sup | The number of pauses whose length was greater than 10 seconds per |
| 10_Ratio_per_min | minute |

The features extraction module 54 is configured to output values for exemplary acoustic features as shown in the table below. It should be appreciated that any number and any combination of such features could be extracted. Acoustic features are those that have been derived from the sonogram data 57.

| Acoustic Feature | Description |
|---|---|
| Loudness | How loud the subject spoke |
| Pitch | the quality of how “high” or “low” the sound is |
| Intonation | Number of intonation events (peaks in a graphical |
| | representation (spectrogram data 57) of pitch) |

The features extraction module 54 is configured to output values for exemplary mapping features as shown in the table below. It should be appreciated that any number and any combination of such features could be extracted. Mapping features are those that have been mapped from the text data 52. Reference to the story in the table below relates to the subject's retelling of a story (or other pre-recorded audio data 29) that has been played to the subject as described heretofore. As such, the features extraction module 54 has access to reference text data relating to the played story for comparison purposes.

| Mapping Feature | Description |
|---|---|
| Synonyms | Any synonyms to the story keywords. |
| Synonyms_ratio | A count of the number of unique synonyms achieved for each |
| | word divided by the total number of words. |
| Synonyms_unique | A count of the number of unique synonyms achieved for each |
| | word. |
| Plurals_Ratio | The ratio that gives an idea of how many plural words were used |
| | in each sentence. |
| Story_Score | Number of story keywords that were detected (according to two |
| | sets of words: 1 and 2 points set of words arranged according to |
| | their complexity). That is, for one set of story words two points |
| | will be awarded to the story score and for another set of story |
| | words just one point will be awarded to the story score. |
| Pronouns_Ratio | The ratio that gives an idea of how many pronouns were used in |
| | each sentence. |
| Present_Progressive_Ratio | The ratio that gives an idea of how many present progressive |
| | phrases were used in each sentence. |
| Grammar_Kernels | A measure of how cohesive each sentence is. Specifically, looking |
| | into subjective and dominant clauses. |
| SpeechRec_Miswritten_Words_Ratio | A ratio that indicates how many words the speech recognition app |
| | incorrectly spelled. |
| Different_Words Count | A Count of the number of unique words that appeared in the text |
| | data 52. |
| Different_Words_Ratio | A ratio that indicates how many different unique words were used |
| | in each sentence. |
| And_Or_Ratio | The ratio of the words: and/or relative to the total number of words |
| | in the text data 52. |
| Low_Frequency_Words_Ratio | A ratio that indicates how many low frequency words were used in |
| | each sentence by comparing words with a library of words that are |
| | infrequently used. |
| Subordinate_Clauses | Count of the number of subordinate clauses that were used in each |
| | sentence. |
| Subordinate_Clauses_Ratio | A ratio that indicates how many subordinate clauses were used in |
| | each sentence. |
| Total_Number_Words | The total number of words that the speech recognition module 50 |
| | transcribed in the text data 52. |
| Mean_Number_Words | The average number of words per utterance that the speech |
| | recognition module 50 transcribed in the text data 52. |

All of these features (acoustic, audio and mapping) are then stored and output as extracted features data 56 for use by the classification system 64. The classification system 64 is further configured to receive spectrogram data 57 as an input, in some embodiments.

In various embodiments, the classification system 64 is configured to receive extracted features data 56, to use at least one machine learning classifier 66, and to output a classification data 68 that can be transformed into diagnosis/screening data 36 representing whether, or a likelihood, that the subject has a language disorder. In embodiments, the classification system 64 is another module of the language disorder diagnostic/screening tool 16. The classification system 64 is configured to use a plurality of different types of machine classifiers 66 to produce plural outputs in the classification data 68, each output representing whether, or a likelihood, of the subject having the language disorder.

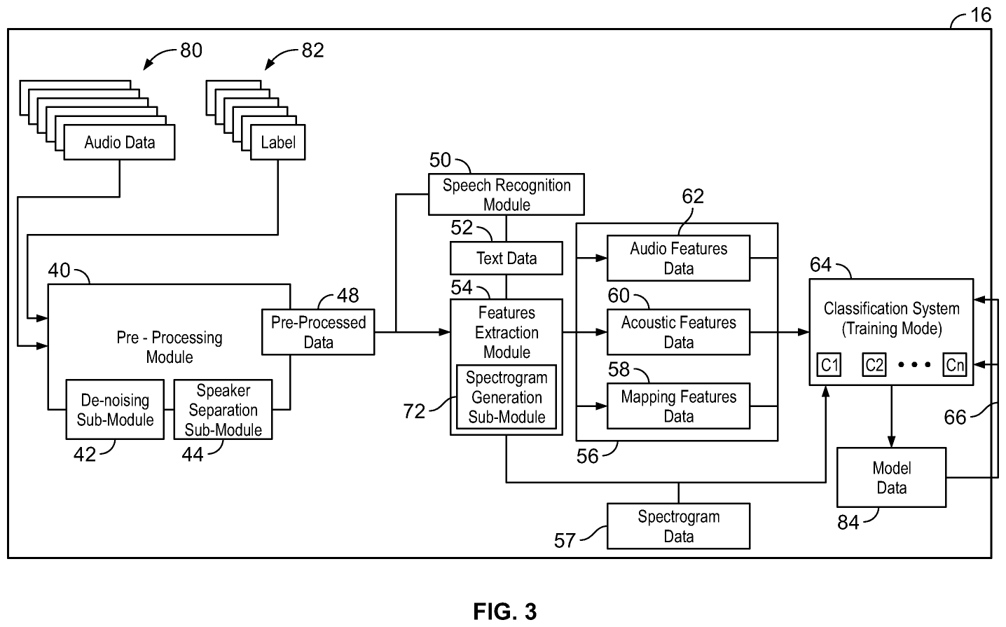

In one embodiment, three different classifiers 66 are included in the classification system 64 to evaluate the extracted features data 56. However, other numbers and types of classifiers are possible (such as two or more different classifiers 66, three or more different classifiers 66, etc.). In one example, the following combination of three classifiers 66 is included: Random Forest, Convolutional Neural Networks (CNN), and linear regression. However, only two of these classifiers 66 could be included in other examples and in any combination. Random Forest, Convolutional Neural Networks (CNN) and linear regression correspond to supervised machine learning approaches. As such, it is envisaged to include two or more different types of machine learning classifiers 66 in the classification system 64. Each of the one or more machine learning classifiers 66 are trained upon a labelled training set, as described further with respect to FIG. 3, so that a training model learns the required parameters for the classifiers 66 to classify the training set. Once parameter optimized, the one or more machine learning classifiers are operable to classify live extracted features data 56. In exemplary embodiments, classification outputs from each classifier are binary (0,1) where “1” corresponds to language disorder and “0” means no language disorder is present (or vice versa). In other embodiments, the outputs of each classifier include three possibilities, namely language disorder, no language disorder and maybe language disorder (e.g. 0, 1 and 2, respectively). However, probability or score-based outputs are also envisaged.

In general, the random forest method is a supervised machine learning method that builds multiple decision trees and merges them together to get a more stable and accurate prediction. To illustrate, a regular decision tree builds a model on what the “best” features are. However, methods such as these are prone to overfitting. Random Forest accounts for this by first building a decision tree from the best features of random subset of features, and then repeats this process for additional subsets of features, resulting in a greater diversity of trees and increasing randomness, which helps to counter the overfitting issue. These trees are then combined to create a classification. In embodiments, the random forest classifier is configured to operate by feeding the extracted features data 56 into a random forest model and creating a classification, in the form of classification data 68, based thereon.

A convolution neural network, CNN, classifier is a deep learning implementation. CNN is configured to receive spectrogram data 57, which includes transformations of pre-processed audio data 48 into spectrogram images. The spectrogram data 57 is input to the CNN which is configured to produce a classification as to whether the subject has a language disorder.

In various embodiments, a linear regression classifier is based on weights which have been assigned by a Speech-therapist to each studied feature in the extracted features data 56. A product of a weight vector (corresponding to the assigned weights for each features) and the extracted feature values (corresponding to the extracted features data 56) is obtained and applied to a linear regression classification function. In some embodiments, the function includes one or more thresholds defining respective classifications. In one embodiment, the function normalizes the product within a cumulative distribution on a 0-100 scale (or some other scale) which indicates, when classification thresholds are applied, whether or not a subject has a language disorder. This normalization process involves, in some embodiments, reference data that has been obtained during training as described below. Specifically, those on the higher half of the scale a have a language disorder (>50), whilst those that score low (0-49) are classified as not having a language disorder. The 0 to 100 normalization score is purely exemplary and other scales could be used. Further, the division of. 50 (or greater than halfway point of range) representing language disorder subjects and the lower half representing no language disorder subjects is provided purely by way of example and other divisions of the scale for classification are possible. A three-way classification is envisaged in other embodiments, whereby one end range of the total score range provides a classification of the subject having a language disorder, another end range provides a classification of the subject not having a language disorder and a middle range corresponds to an unclear state as to whether the subject has a language disorder.

In various embodiments, each of the classifiers 66 is trained using different training models. FIG. 3 illustrates the language disorder diagnostic/screening tool 16 in a training mode. The modules are largely the same as those described with respect to FIG. 2. However, the language disorder diagnostic/screening tool 16 operates the classification system 64 so as to generate and/or optimize parameters of each classifier 66. In particular, the classification system 64 generates model data 84 that is fed back to the classifiers 66 and used thereby for subsequent classifying or further training and parameter optimization. For training, a library of audio data 80 that has been labelled (by a human in some embodiments) or is otherwise accompanied by reference data is fed through the language disorder diagnostic/screening tool 16. In embodiments, the library of audio data 80 is pre-processed by pre-processing module 40 and spectrogram data 57 and extracted features data 56 is generated as described above with respect to FIG. 2. The classification system 64 in training mode uses true/verified labels 82 for the audio data 80 as reference data for generating and/or optimizing model data 84 for each of classifiers 66, as will be described with respect to example classifiers in the following. Labels 82 are, in some embodiments, true/validated labels associated with each audio data file 80. The labelling may be performed by a speech therapist.

In embodiments, and with continued reference to FIG. 3, the audio data 80 and associated labels 82 (true/validated labels 82) include pre-existing libraries that have been labelled by a speech therapist or audio files that have been labelled by the language disorder diagnostic/screening tool 16 previously. In embodiments, the labels 82 include a vector representing two or more classification states of no language disorder, language disorder and maybe language disorder (e.g. 0, 1 and 2, respectively). The training mode is configured to generate (and continuously update) a model for each classifier, embodied by model data 84. Details of each training process are dependent on the type of classifier 66. Since different types of classifiers are implemented, plural different training processes will be followed.

In one example, a CNN classifier is trained. The CNN classifier in training mode takes processed audio data 80 (processed per modules 40, 50 and 54 as described heretofore), which has been transformed into spectrogram data 57, and associated labels 82 as inputs to generate and optimize the CNN model according to known techniques. The CNN classifier will be retrained periodically for optimization as new audio data is recorded and the associated labels 82 generated. Trained CNN parameters are incorporated into model data 84 for subsequent use by the CNN classifier.

In another example, training of the linear regression classifier uses features extracted from the library of audio data 80 in the form of extracted features data 56. A set of averaged features values are obtained for extracted features data 56 from audio data 80 associated with a no language disorder label 82 for that subject. An average of feature values for all of audio data 80 from subjects labelled as not having a language disorder. The thus obtained vector of average feature values, which forms reference data as described above with respect to the linear regression classifier, for no language disorder subjects is used for subsequent inference (in normal operating mode of the language disorder diagnostic/screening tool 16). The linear regression classifier will be retrained periodically to optimize model data 84. The vector of average feature values forms reference data for subsequent use by the linear regression classifier and is incorporated into model data 84.

In an example detailed operation of the linear regression classifier, a ratio of each feature value extracted from the audio data 34 of a subject to be assessed with respect to the corresponding feature value in the vector of average features values is obtained. The resulting ratio value is normalized onto a scale (e.g. a scale from 0 or 1 to n, wherein n is any value from, for example, 3 to 100). The normalized or scaled value is multiplied by a percentage weight factor (provided, in examples, by a human specialist in the field), where the weight represents the perceived importance of each respective feature. The products are summed and normalized and subsequently categorized, based on thresholds, into one of the possible classification outputs. These classification outputs include, in examples, no language disorder, language disorder and optionally possible language disorder.

In one example of training a random forest classifier extracted features data 56 is taken from each of a library of existing audio data 80. The resulting set of extracted features data 56, along with the corresponding labels 82, are inputs to train the random forest classifier in training model. The training results in a model being created that is included in model data 84. The resulting model data 84 is used in the random forest classifier for subsequent classifications in normal operating mode.

Referring back to FIG. 2, and in accordance with various embodiments, the language disorder diagnostic/screening tool 16 includes a classification combination module 70 configured to receive plural classifications from respective classifiers 66, which classifications are included in the classification data 68. The classification combination module 70 is configured to combine classifications in the classification data 68 so as to provide diagnosis/screening data 36 representing a single classification as to whether the subject has a language disorder. The classification combination module 70 is configured to apply different weights to at least two classifications from different classifiers 66 in one embodiment. The weights can be determined and updated based on the overall label provided for each subject by an expert speech therapist. The classification combination module 70 is configured to use logistic regression to combine the classification outputs in one example algorithmic method. An exemplary algorithm for the weighted combination includes w1*c1+w2*c2+ . . . wn*cn, where w1, w2 . . . wn are the weights for each classifier and c1, c2 . . . cn are the classification scores from respective classifiers 66 included in the classification data 68. Based on the combined score from the classification combination module 70, an output result of “no language disorder”, “language disorder” and optionally an intermediate “possible language disorder” classification result could be output by the language disorder diagnostic/screening tool 16 in the form of diagnostic/screening data 36. In some embodiments, each classifier 66 outputs a binary classification in classification data 68 and the classification combination sub-module 70 outputs a tertiary classification (i.e. one out of three possibilities) in diagnosis or screening data 36.

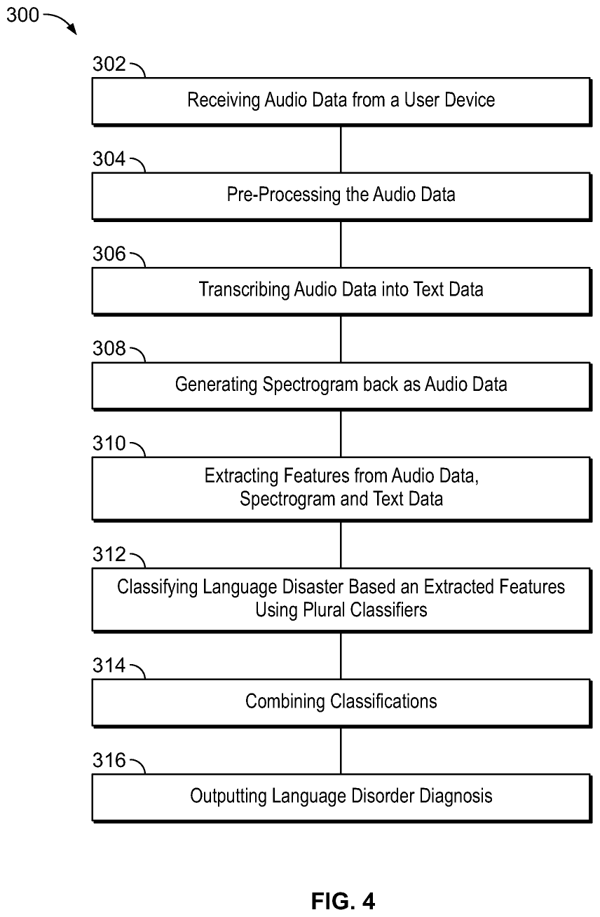

FIG. 4 provides a flowchart for processor executed steps of a method 300 for diagnosing a language disorder, in accordance with various embodiments. The method 300 is, in some embodiments, carried out by the processor 30 of the server 14 unless stated otherwise. In particular, the processor 30 executes various software instructions including those described with respect to the modules of the language disorder diagnostic/screening tool 16 of FIG. 2.

Continuing to refer to FIG. 4, the method 300 includes step 302 of receiving audio data 34 from a user device 12. In embodiments, the audio data 34 is provided by internet communication or other far communication method through communication channels 35. As has been heretofore explained, the audio data 34 is recorded by audio recording device 20 of user device 12 and includes, in some embodiments, retelling of a story (or other played pre-recorded audio data 29) by a subject that has been told the story through the audio play device 22 of the user device 12.

In the embodiment of FIG. 4, the method 300 includes step 304 of pre-processing audio data 34. The pre-processing includes applying denoising and speaker separation algorithms, per denoising and speaker separation sub-modules 42, 44 of FIG. 2, to provide clear, pre-processed audio data 48, including substantially only the subject's speech, with background noise and any other speakers removed.

In the exemplary embodiment of FIG. 4, the method 300 includes the step 306 of transcribing, via speech recognition module 50 of FIG. 2, the pre-processed audio data 48 into text data 52. Further, a spectrogram is generated, via spectrogram generation sub-module 72, based on the pre-processed audio data 48.

In accordance with various embodiments, the method 300 includes step 310 of extracting features from a combination of at least two of text data 52, spectrogram data 57 and pre-processed audio data 48. As has been explained herein, extracted features data 56 includes audio features data 62, acoustic features data 60 and mapping features data 58. Audio features data 62 include features extracted from pre-processed audio data 48 including various parameters associated with time of pauses in subject speech and time of utterances in subject speech. Acoustic features data 60 includes features extracted from spectrogram data 57 including parameters associated with pitch, loudness and intonation. Audio features data 60 includes features extracted from text data 52 including parameters associated with words used.

In embodiments, the method 300 includes classifying a language disorder based on the extracted features data 56 using plural classifiers 66 including at least one machine learning classifier. At least one or some of the classifiers 66 have been trained based on a library of audio data 80 and associated language disorder labels 82. In embodiment, as discussed in the foregoing, the classifiers 66 of a classification system 64 include at least one of a CNN classifier, a random forest classifier and a linear regression classifier. In one embodiment, the CNN classifier is configured to classify based on spectrogram data 57. In one embodiment, the linear regression classifier is configured to use thresholds to classify the subject based on values of the extracted features data 56, which includes acoustic features data 60 (taken from spectrogram data 57), mapping features data 58 and audio features data 62.

In embodiments, the method 300 includes step 314 of outputting classifications from respective classifiers 66. The classifications are included in classification data 68 received by classification combination module 70. Various classification combination algorithms are possible including weighted average-based combinations or weighted sum. The weights are reference values optionally set by a speech therapy expert.

In accordance with various embodiments, the method 300 includes step 316 of outputting a language disorder diagnosis/screening based on, or corresponding to, the combined classifications from step 314. The language disorder diagnosis/screening data 36 can include binary or three-way states including no language disorder, language disorder and optionally uncertain language disorder. In other embodiments, the language disorder diagnosis/screening data 36 includes a scaled score (e.g. on a scale of 1 to 10 or 1 to 100). The language disorder diagnosis/screening data 36 is sent to the user device over communications channels 35 for display on display device 26. The language disorder diagnostic/screening tool application 18 is configured in some embodiments to display next steps for the subject based on the diagnosis/screening data 36. Such next steps include to seek further consultation with a human speech therapy expert when uncertain language disorder or language disorder diagnoses are returned. In some embodiment, the language disorder diagnosis/screening data 36 is sent additionally or alternatively to a device of a speech therapist or an associated institution.

While at least one exemplary aspect has been presented in the foregoing detailed description of the invention, it should be appreciated that a vast number of variations exist. It should also be appreciated that the exemplary aspect or exemplary aspects are only examples, and are not intended to limit the scope, applicability, or configuration of the invention in any way. Rather, the foregoing detailed description will provide those skilled in the art with a convenient road map for implementing an exemplary aspect of the invention. It being understood that various changes may be made in the function and arrangement of elements described in an exemplary aspect without departing from the scope of the invention as set forth in the appended claims.

What is claimed is:

1. A language disorder diagnostic/screening method, the method comprising:
receiving audio data including speech of a subject;
transcribing, via at least one processor, the audio data to provide text data;
extracting, via at least one processor, speech and language features from the text data and from the audio data;
evaluating the extracted features using a classification system to diagnose/screen whether the subject has a language disorder, wherein the classification system includes at least one machine learning classifier; and
outputting the diagnosis/screening.

2. The method of claim 1, wherein the language disorder is Developmental Language Disorder.

3. The method of claim 1, wherein the classification system includes a plurality of classifiers, the method comprising combining, via at least one processor, classification outputs from each of the plurality of classifiers.

4. The method of claim 3, wherein classification outputs from each of the plurality of classifiers is combined using a different weighting.

5. The method of claim 1, wherein the classification system includes a random forest classifier.

6. The method of claim 1, wherein the classification system includes a convolution neural network.

7. The method of claim 1, wherein the classification system includes a linear regression classifier.

8. The method of claim 1, wherein the classification system includes at least two of a random forest classifier, a linear regression classifier and a convolutional neural network.

9. The method of claim 1, comprising transforming, via at least one processor, the audio data into a spectrogram and evaluating the extracted features and the spectrogram images using the classification system to diagnose/screen whether the subject has a language disorder.

10. The method of claim 1, comprising pre-processing of the audio data prior to extracting speech features, wherein pre-processing comprises at least one of denoising and speaker separation.

11. The method of claim 1, wherein the extracted features includes at least one of audio features, acoustic features and mapping features derived from the text data.

12. The method of claim 11, wherein audio features include features related to time of speech by subject in the audio data and/or time of pauses by the subject in the audio data, wherein the acoustic features include loudness, pitch and/or intonation and wherein the mapping features include features related to variety of language in the text data, sophistication of language in the text data and/or grammar related features derived from the text data.

13. The method of claim 11, wherein the audio features includes at least one of number of pauses in the audio data, number of pauses per minute in the audio data, maximum length of utterances in the audio data, average length of utterances in the audio data, total length of time of speech of the subject in the audio data, maximum length of a pause in the audio data, ratio of maximum length of a pause and total length of time of speech of subject in the audio data, average length of pauses in the audio data, ratio of average length of pauses and total speech length, number of pauses having a length greater than five seconds in the audio data, number of pauses having a length greater than ten seconds in the audio data, the number of pauses per minute greater than ten seconds.

14. The method of claim 11, wherein the acoustic features are extracted from a spectrogram of the audio data.

15. The method of claim 11, wherein the acoustic features include at least one of loudness, pitch and intonation of the speech of the subject.

16. The method of claim 11, wherein the mapping features include at least one of synonyms to story keywords, a count of the number of unique synonyms achieved for each word divided by the total number of words, a count of the number of unique synonyms achieved for each word, a ratio representing how many plural words were used in the sentence, number of story keywords that were detected, a ratio representing how many pronouns were used per sentence, a ratio representing how many present progressive phrases were used per sentence, a measure of how cohesive the sentence is based on subjective and dominant clauses, a ratio that indicates how many words are incorrectly spelled in the text data, a count of the number of unique words that appeared in the list, a ratio that indicates how many different unique words were used per sentence, the ratio of the words: and/or in the document, a ratio that indicates how many low frequency words were used in the sentence, a count of the number of subordinate clauses that were used in the sentence, a ratio that indicates how many subordinate clauses were used in the sentence, a total number of words in the text data and an average number of words per utterance in the text data.

17. A language disorder diagnosis/screening tool, comprising:
at least one processor configured to receive audio data including speech of a subject;
at least one processor configured to transcribe the audio data to provide text data;
at least one processor configured to extract speech features from the text data and from the audio data;
a classification system configured to evaluate the extracted features to diagnose/screen whether the subject has a language disorder, wherein the classification system includes at least one machine learning classifier; and
at least one processor configured to output the diagnosis/screening.

18. The language disorder diagnosis/screening tool of claim 17, wherein at least one of:
the audio data is recorded on a user device;
the output of the diagnosis/screening is to a user device; and
the classification system is at a server remote from a user device or the classification system is at the user device.

19. The language disorder diagnosis/screening tool of claim 18, wherein the language disorder is Developmental Language Disorder DLD.

20. At least one software application configured to be run by at least processor to cause:
transcribing received audio data to provide text data;
extracting speech and language features from the text data and from the audio data;
evaluating the extracted features using a classification system to diagnose/screen whether the subject has a language disorder, wherein the classification system includes at least one machine learning classifier; and
outputting the diagnosis/screening.
